(19) United States
(12) Patent Application Publication
(10) Pub. No.: US 2019/0150711 A1
(43) Pub. Date: May 23, 2019

(54) Endoscope Apparatus

(71) Applicant: Issa Technology Co., Ltd., Taoyuan City (TW)

(72) Inventors: Chi-Wei Chiu, Taoyuan City (TW); Rong-Jhe Chen, Taoyuan City (TW)

(21) Appl. No.: 15/953,570

(22) Filed: Apr. 16, 2018

(30) Foreign Application Priority Data
Nov. 17, 2017 (TW) 106139858

(51) Int. Cl. A61B 1/00; H04N 5/225; A61B 1/005; A61B 1/04; A61B 1/06

(52) U.S. Cl. CPC A61B 1/00071; H04N 5/2256; H04N 5/2254; A61B 1/0661; A61B 1/005; A61B 1/04; A61B 1/00105

(57) ABSTRACT

An endoscope apparatus includes a flexible print circuit board (FPC), a light-emitting module and an image-capturing module. The FPC has a first surface and a second surface and divided to a first installing portion, a second installing portion and a first bridge portion. The first portion has an opening. The second installing portion is located opposite to the first installing portion. The first bridge portion is connected between the first installing portion and the second installing portion. The light-emitting module has a plurality of light sources, which are disposed on the first surface of the first installing portion. The image-capturing module is disposed on the second surface of the second installing portion of the FPC. The image-capturing module has an image sensing area, which is disposed corresponding to the opening of the first installing portion.

### CROSS REFERENCE TO RELATED APPLICATIONS

This Non-provisional application claims priority under 35 U.S.C. § 119(a) on Patent Application No. 106139858 filed in Republic of China on Nov. 17, 2017, the entire contents of which are hereby incorporated by reference.

### BACKGROUND

### 1. Technical Field

This invention relates to an endoscope apparatus, and in particular, to a small-caliber endoscope apparatus.

### 2. Description of Related Art

An endoscope is an illuminated optical, typically slender and tubular instrument used to look deep into the body and used in procedures called an endoscopy. Since the endoscopes can be inserted directly into the organ, the endoscopy procedure uses an endoscope to examine the interior of a hollow organ or cavity of the body. Endoscopes are also a tool helpful in the architectural engineering and electronic equipment. For example, the planning and architectural community use architectural endoscopy for pre-visualization of scale models of proposed buildings and cities. For non-medical use, similar instruments are also called borescopes.

An endoscope usually consists of a lens module transmitting image to a screen for image capture, a sleeve lens holder to fix the lens module therein, a circuit board electrically connected to the lens module, and a tube accommodating the lens module, the lens holder and the circuit board therein. The lens module is locked and contained within the traditional sleeve lens holder. Accordingly, downsizing of the endoscope apparatus is limited by these components, including the lens module, the lens holder and the circuit board. However, a lens module with a better resolution is usually bigger in size. The tube of a lens module with a 720P resolution is about 5.5 millimeter (mm) in diameter or more.

Related to endoscope size, the requirement of inserting the endoscope tube into human body or other equipment limits the dimensions and diameter of the endoscope apparatus. The finer the endoscope apparatus is, the more narrow conditions the endoscope apparatus can be applied to. Trials reports that patient discomfort is lower when small-caliber endoscopes are used.

### SUMMARY OF THE INVENTION

It is therefore an object of the present invention to provide a small-caliber endoscope apparatus with fewer elements.

To achieve the above, the present invention provides an endoscope apparatus including a flexible print circuit board (FPC), a light-emitting module and an image-capturing module. The flexible print circuit board has a first surface and a second surface opposite to the first surface. The flexible print circuit board includes a first installing portion having a first opening, a second installing portion located opposite to the first installing portion, and a first bridge portion connecting the first installing portion and the second installing portion. The light-emitting module is located on the first surface of the flexible print circuit board in the first installing portion. The image-capturing module is located on the second surface of the flexible print circuit board in the second installing portion. The image-capturing module has an image sensing area, and the image sensing area is located corresponding to the first opening of the first installing portion.

In another embodiment of the present invention, the light-emitting module having light sources around the first opening on the first surface in the first installing portion.

In another embodiment of the present invention, the flexible print circuit board further includes a third installing portion and a second bridge portion connecting the second installing portion and the third installing portion.

In another embodiment of the present invention, the third installing portion is not parallel to the first installing portion and the second installing portion.

In another embodiment of the present invention, the first installing portion and the second installing portion of the flexible print circuit board are circle sheets or round sheets.

In another embodiment of the present invention, the endoscope apparatus includes a tube, and the first installing portion, the second installing portion, the first bridge portion and the second bridge portion of the flexible print circuit board are all disposed in the tube.

In another embodiment of the present invention, the image-capturing module is directly contacted or assembled to the second surface of the flexible print circuit board.

In another embodiment of the present invention, a diameter of the tube, a diameter of the first installing portion and a diameter of the second installing portion are all less than 5 mm.

Since the image-capturing module and the light-emitting module of the endoscope apparatus are installed directly on the flexible print circuit board in the present invention, when the flexible print circuit board is folded in the tube, all components can be placed within the tube. Unlike the traditional endoscope having a sleeve lens holder to fix the image-capturing module onto the circuit board, the image-capturing module of the present invention is directly fixed on the flexible print circuit board without the lens holder. Thus, a small-caliber endoscope is provided with a finer tube in diameter.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The parts in the drawings are not necessarily drawn to scale, the emphasis instead being placed upon clearly illustrating the principles of at least one embodiment. In the drawings, like reference numerals designate corresponding parts throughout the various diagrams, and all the diagrams are schematic.

FIG. 1 is an explosion diagram illustrating an endoscope apparatus according to one embodiment of the present invention.

FIG. 2 is a side view of some components of the endoscope apparatus according to the embodiment of the present invention.

FIG. 3 is a top view of some components of the endoscope apparatus on the second surface of the flexible print circuit board according to the embodiment of the present invention.

FIG. 4 is a bottom view of some components of the endoscope apparatus on the first surface of the flexible print circuit board according to the embodiment of the present invention.

FIG. 5 is a perspective schematic diagram illustrating the endoscope apparatus according to the embodiment of the present invention.

### DETAILED DESCRIPTION

Reference will now be made to the drawings to describe various inventive embodiments of the present disclosure in detail, wherein like numerals refer to like elements throughout.

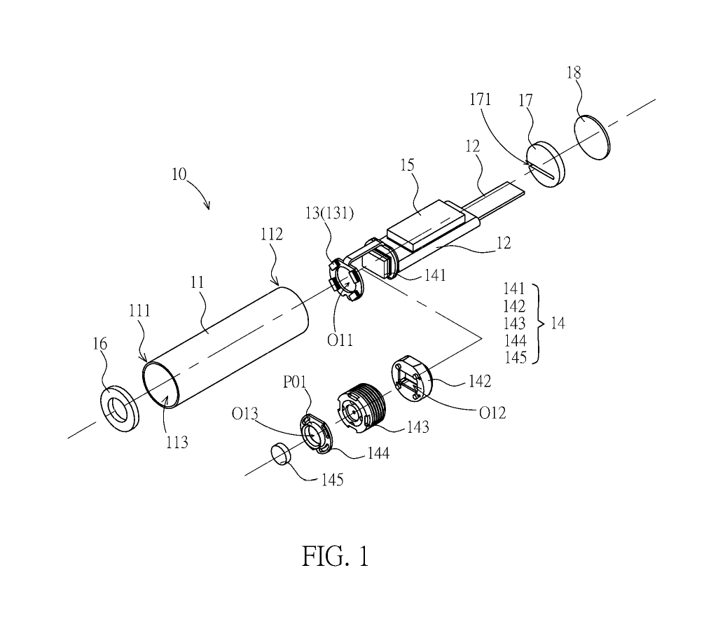

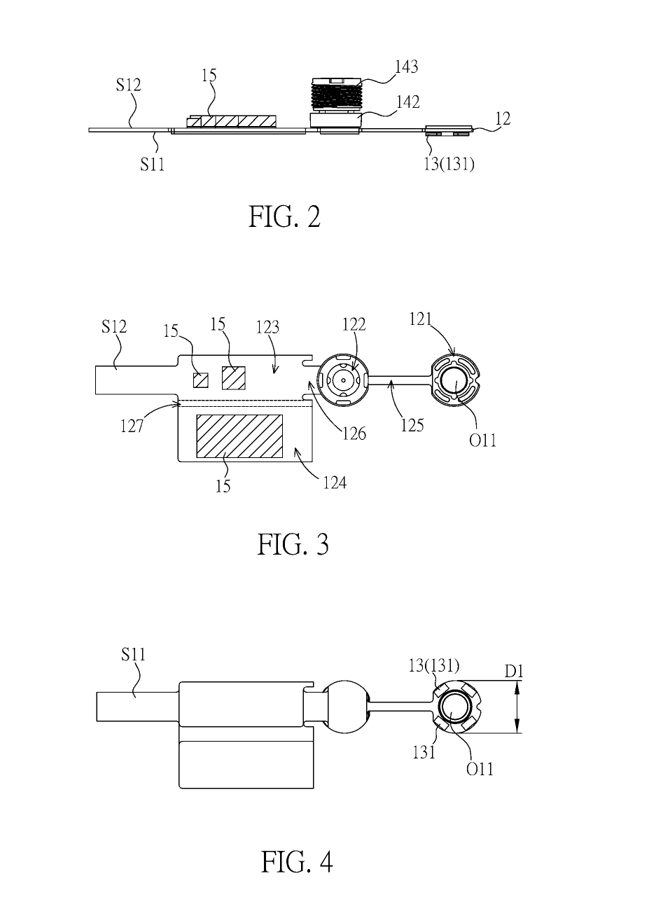

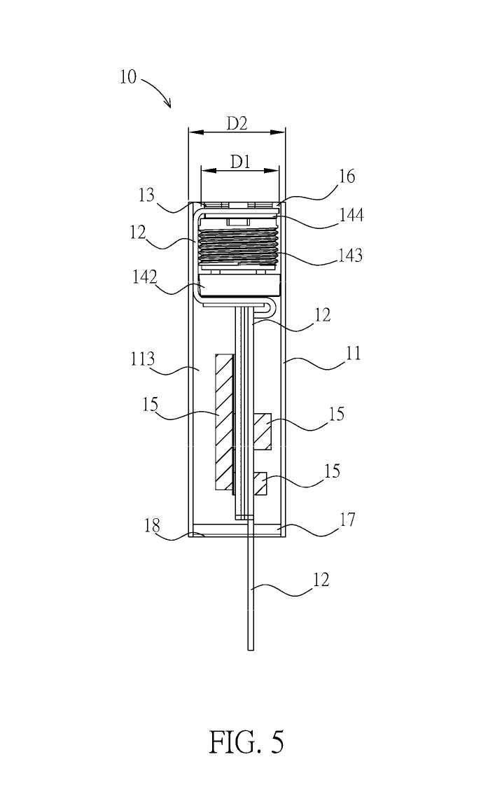

Referring to FIG. 1 through FIG. 5, FIG. 1 is an explosion diagram illustrating an endoscope apparatus 10 according to one embodiment of the present invention; FIG. 2 is a side view of some components of the endoscope apparatus 10 according to the embodiment of the present invention; FIG. 3 is a top view of some components of the endoscope apparatus 10 on the second surface S12 of the flexible print circuit board 12 according to the embodiment of the present invention; FIG. 4 is a bottom view of some components of the endoscope apparatus 10 on the first surface of the flexible print circuit board 12 according to the embodiment of the present invention; and FIG. 5 is a perspective schematic diagram illustrating the endoscope apparatus 10 according to the embodiment of the present invention.

As shown in FIG. 1, an endoscope apparatus 10 includes a tube 11, a flexible print circuit board (FPC) 12, a light-emitting module 13, an image-capturing module 14, numbers of the electronic components 15, a first adhesive component 16, a bottom cover 17 and a second adhesive component 18 in this embodiment.

The tube 11 has a first terminal 111, a second terminal 112 opposite to the first terminal 111, and an accommodate space 113 located between the first terminal 111 and the second terminal 112. The accommodate space 113 is designed to accommodate the flexible print circuit board 12, the light-emitting module 13, the image-capturing module 14, the electronic components 15, the first adhesive component 16, the bottom cover 17 and the second adhesive component 18. The tube 11 may be made from metal or plastic, or may include other mixture, such as stainless steel or plastic steel. The tube 11 may be rigid or flexible as required.

As shown in FIG. 2 through FIG. 4, the flexible print circuit board 12 has a first surface S11 and a second surface S12 opposite to the first surface S11. The flexible print circuit board 12 can be divided into a first installing portion 121, a second installing portion 122, a third installing portion 123, a fourth installing portion 124, a first bridge portion 125, a second bridge portion 126 and a third bridge portion 127. The first bridge portion 125 is connected between the first installing portion 121 and the second installing portion 122; the second bridge portion 126 is connected between the second installing portion 122 and the third installing portion 123; and the third bridge portion 127 is connected between the third installing portion 123 and the fourth installing portion 124.

The first installing portion 121 is a circle sheet or a round sheet, and has a first opening O11 in the center. The second installing portion 122 is also a circle sheet or a round sheet. The diameter D1 (or the largest length) of the first installing portion 121 and the second installing portion 122 is substantially less than 5 mm, and is preferred between 3 mm through 4 mm.

As shown in FIG. 3 through FIG. 5, when the third bridge portion 127 is bent toward the first surface S11, the third installing portion 123 overlaps and contacts the fourth installing portion 124. In case the third installing portion 123 covers the fourth installing portion 124, the first surface S11 of the third installing portion 123 turns toward the first surface S11 of the fourth installing portion 124. After that, as shown in FIG. 5, when the second bridge portion 126 is bent toward the first surface S11 for 90 degrees, the second installing portion 122 covers the side surface of the third installing portion 123.

The first installing portion 121 is parallel to the second installing portion 122, and the third installing portion 123 is not parallel to the first installing portion 121 and the second installing portion 122. In particular, the first surface S11 or the second surface S12 within the first installing portion 121 is parallel to the first surface S11 or the second surface S12 within the second installing portion 122; and the first surface S11 or the second surface S12 within the third installing portion 123 is not parallel to the first surface S 11 or the second surface S12 within the first installing portion 121 and the second installing portion 122.

Specifically speaking, the first surface S11 or the second surface S12 within the third installing portion 123 is vertical to the first surface S11 or the second surface S12 within the first installing portion 121 and the second installing portion 122. The angle between the third installing portion 123 and the first installing portion 121 (or the second installing portion 122) is about 70 through 110 degrees, which can be adjusted according to size of the accommodate space 113 of the tube 11 in other embodiment.

As shown in FIG. 2 and FIG. 4, the light-emitting module 13 includes numbers of the light sources 131, which are individually located on the first surface S11 of the first installing portion 121. The light sources 131 may be light-emitting devices (LEDs) or organic light-emitting diodes (OLEDs), and are not limited therein. The light sources 131 are located around the first opening O11 on the first surface S11 in the first installing portion 121, and are electrically connected to the first surface S11 of the flexible print circuit board 12 in the first installing portion 121. The light-emitting module 13 is applied to illuminate the organ or object in the endoscopy procedure. In other embodiment, the light sources 131 may be omitted, and the light-emitting module 13 may include an optical fiber system to direct the light.

As shown in FIG. 1 through FIG. 3, the image-capturing module 14 includes an image sensor 141, a lens base 142, a lens module 143 and a fixture 144. The image sensor 141 may be any kind of image sensor, such as a charge coupled device (CCD) or a complementary metal-oxide semiconductor (CMOS). The image sensor 141 is located on and electrically connected to the second surface S12 of the flexible print circuit board 12 in the second installing portion 122. The lens base 142 has a second opening O12. The image sensor 141 is encircled by the lens base 142, and the second opening O12 of the lens base 142 exposes the image sensing area of the image sensor 141. The lens module 143 is located on the lens base 142, so the images can be captured in the image sensing area of the image sensor 141. It is to be noted that the image sensor may be directly contacted to the second surface of the flexible print circuit board to reduce the whole size. Moreover, in another embodiment, the image sensor may be assembled to the second surface of the flexible print circuit board through the connector or the glue, for example, UV glue.

The fixture 144 is located on the lens module 143, and has a third opening O13. The fixture 144 includes a protrusion P01 surround the third opening O13 away from the lens module 143. A transparent protection cap 145 is located in the protrusion P01 of the fixture 144 corresponding to the third opening O13.

The electronic components 15 are located on and electrically connected to the third installing portion 123 and the fourth installing portion 124 of the flexible print circuit board 12. The electronic components 15 may include passive electronic components or active electronic components, such as capacitors or the controllers.

Since the flexible print circuit board 12 is flexible, components can be stacked in the endoscope apparatus 10. As shown in FIG. 3 through FIG. 5, when the first bridge portion 125 of the flexible print circuit board 12 is bent toward the second surface S12, the first installing portion 121 overlaps the second installing portion 122, and the second surface S12 of the first installing portion 121 turns toward the second surface S12 of the second installing portion 122. As the flexible print circuit board 12 is folded, the image sensing area of the image-capturing module 14 is corresponding to the first opening O11 of the first installing portion 121 and the lens module 143. In addition, the protrusion P01 of the fixture 144 is encircled by the first opening O11 of the first installing portion 121 to fix the relative locations of the light-emitting module 13 and the image-capturing module 14.

As shown in FIG. 1 and FIG. 5, the first adhesive component 16 is located at the first terminal 111 of the tube 11 to seal the first terminal 111. The first adhesive component 16 can ensure the internal components inside the tube 11, and blocks moisture and impurities outside the tube 11. The first adhesive component 16 may be made from ultraviolet glue, the optical clear adhesive or any other transparent adhesive in this embodiment.

The bottom cover 17 is located at the second terminal 112 of the tube 11. The bottom cover 17 has a trench 171 to allow the penetrating of the flexible print circuit board 12. The second adhesive component 18 is located outside the bottom cover 17 to fix the bottom cover 17 and the tube 11. The second adhesive component 18 can ensure the internal components inside the tube 11, and blocks moisture and impurities outside the tube 11.

As shown in FIG. 5, since the image-capturing module 14 is directly connected to the flexible print circuit board 12, and the folded flexible print circuit board 12 fixes the location of the image-capturing module 14, a small-caliber endoscope apparatus 10 is provided with a finer tube 11 in diameter without additional lens holder. The diameter D2 of the tube 11 is substantially less than 5 mm, and is preferred between 3.5 mm through 4.5 mm.

Please referring to the above descriptions and drawings, a method of assembling the endoscope apparatus 10 is briefly described as follows. First, the flexible print circuit board 12 is provided in a step 1. Subsequently, the light-emitting module 13 is placed on the first surface S11 of the flexible print circuit board 12 in the first installing portion 121 in a step 2. Next, the image-capturing module 14 is placed on the second surface S12 of the flexible print circuit board 12 in the second installing portion 122 in a step 3. Thereafter, the electronic components 15 are individually placed on the second surface S12 of the flexible print circuit board 12 in the third installing portion 123 and the fourth installing portion 124 in a step 4. Next, the third bridge portion 127 is bent in a step 5. The first surface S 11 of the third installing portion 123 is turned toward the first surface S11 of the fourth installing portion 124, until the third installing portion 123 overlaps the fourth installing portion 124. Thereafter, the first bridge portion 125 is bent in a step 6. The second surface S12 of the first installing portion 121 is turned toward the second surface S12 of the second installing portion 122, until the first opening O11 of the first installing portion 121 aligns the image sensing area of the image-capturing module 14.

Next, the second bridge portion 126 is bent in a step 7. The second installing portion 122 is turned toward its first surface S11, until the first surface S11 of the second installing portion 122 is vertical to the first surface S11 of the third installing portion 123. The endoscope apparatus 10 is semi-finished in the step 7. The semi-finished endoscope apparatus 10 is placed in the tube 11 in a step 8. Thereafter, the two terminals 111, 112 of the tube 11 are sealed to form the endoscope apparatus 10. It is noticed that, the above-mentioned assembly steps may be adjusted or combined in other embodiment.

More in detail, the step 3 of providing the image-capturing module 14 on the flexible print circuit board 12 includes the following steps. First, the image sensor 141 is electrically connected to the second surface S12 of the flexible print circuit board 12 in the second installing portion 122 in a step 3-1. Subsequently, the lens base 142 is provided to hold the image sensor 141 in a step 3-2. Next, the lens module 143 is provided on the lens base 142 corresponding to the image sensor 141 in a step 3-3. Thereafter, the fixture 144 is provided on the lens module 143 in a step 3-4. Finally, the transparent protection cap 145 is provided on the fixture 144 corresponding to the lens module 143 in a step 3-5. The steps of providing the image-capturing module 14 are not limited to the above description, and may be adjusted in other embodiment.

In summary, since the image-capturing module and the light-emitting module of the endoscope apparatus are installed directly on the flexible print circuit board in the present invention, all components can be disposed within the tube when the flexible print circuit board is folded in the endoscope apparatus. Unlike the traditional endoscope having a sleeve lens holder to fix an image-capturing module onto the circuit board, the image-capturing module of the present invention is directly disposed on the flexible print circuit board without the lens holder. Since size of the endoscope apparatus is no more limited by the lens holder, the tube size is substantially designed to fit the image-capturing module, and a small-caliber endoscope is provided with a finer tube in diameter.

Even though numerous characteristics and advantages of certain inventive embodiments have been set out in the foregoing description, together with details of the structures and functions of the embodiments, the disclosure is illustrative only. Changes may be made in detail, especially in matters of arrangement of parts, within the principles of the present disclosure to the full extent indicated by the broad general meaning of the terms in which the appended claims are expressed.

What is claimed is:

1. An endoscope apparatus, comprising:
a flexible print circuit board, the flexible print circuit board having a first surface and a second surface opposite the first surface, the flexible print circuit board comprising:
a first installing portion having a first opening;
a second installing portion disposed opposite to the first installing portion; and
a first bridge portion connecting the first installing portion and the second installing portion;
a light-emitting module disposed on the first surface of the flexible print circuit board in the first installing portion; and
an image-capturing module disposed on the second surface of the flexible print circuit board in the second installing portion, the image-capturing module having an image sensing area corresponding to the first opening of the first installing portion.

2. The endoscope apparatus of claim 1, wherein the light-emitting module comprises a plurality of light sources on the first surface around the first opening in the first installing portion.

3. The endoscope apparatus of claim 1, wherein the flexible print circuit board further comprises:
a third installing portion; and
a second bridge portion connecting the second installing portion and the third installing portion.

4. The endoscope apparatus of claim 3, wherein the third installing portion is not parallel to the first installing portion and the second installing portion.

5. The endoscope apparatus of claim 4, wherein the third installing portion is vertical to the first installing portion and the second installing portion.

6. The endoscope apparatus of claim 1, wherein the image-capturing module comprises:
an image sensor disposed on the second surface of the flexible print circuit board in the second installing portion, the image sensor having the image sensing area;
a lens base disposed on the image sensor, the lens base having a second opening exposing the image sensing area of the image sensor;
a lens module disposed on the lens base corresponding to the second opening; and
a fixture disposed on the lens module, and the fixture having a third opening corresponding to the second opening.

7. The endoscope apparatus of claim 6, wherein the image sensor is directly contacted to the second surface of the flexible print circuit board.

8. The endoscope apparatus of claim 6, wherein the image sensor is assembled to the second surface of the flexible print circuit board.

9. The endoscope apparatus of claim 1, wherein the first installing portion and the second installing portion of the flexible print circuit board are circle sheets or round sheets.

10. The endoscope apparatus of claim 9, wherein a diameter of the first installing portion or a diameter of the second installing portion is between 3 millimeter (mm) and 4 mm.

11. The endoscope apparatus of claim 1, further comprising a tube, wherein the first installing portion, the second installing portion and the first bridge portion of the flexible print circuit board are all disposed in the tube.

12. The endoscope apparatus of claim 11, wherein a diameter of the tube is between 3.5 mm and 4.5 mm.

13. The endoscope apparatus of claim 1, wherein the image-capturing module is directly contacted to the second surface of the flexible print circuit board.

14. The endoscope apparatus of claim 1, wherein the image-capturing module is assembled to the second surface of the flexible print circuit board.
